(19) United States
(12) Patent Application Publication
(10) Pub. No.: US 2018/0106774 A1
(43) Pub. Date: Apr. 19, 2018

(54) SENSOR ARRAY, MANUFACTURING METHOD THEREOF, AND SENSING METHOD

(71) Applicant: Winbond Electronics Corp., Taichung City (TW)

(72) Inventors: Yu-Hsuan Ho, Taichung City (TW); Yi-Der Wu, Taichung City (TW); Yu-Ming Chen, Taichung City (TW)

(73) Assignee: Winbond Electronics Corp., Taichung City (TW)

(21) Appl. No.: 15/585,170

(22) Filed: May 3, 2017

(30) Foreign Application Priority Data
Oct. 19, 2016 (CN) 201610908808.9

(51) Int. Cl. G01N 33/00; G01N 27/12

(52) U.S. Cl. CPC G01N 33/0031; G01N 27/12

(57) ABSTRACT

A sensor array includes a circuit board, a plurality of first sensing units, and at least one second sensing unit. The circuit board has an upper surface and a lower surface that are opposite to each other. The first sensing units are located on the upper surface of the circuit board. The first sensing units include a plurality of first electrodes and a plurality of sensing material layers. The sensing material layers are respectively located on surfaces of the first electrodes, and the sensing material layers are manufactured through applying a non-contact printing method. The second sensing unit is located on the upper surface of the circuit board. The second sensing unit includes a second electrode separated from the first electrodes. The sensing material layers respectively cover the surfaces of the first electrodes, and the second electrode is exposed to an atmospheric environment.

### CROSS-REFERENCE TO RELATED APPLICATION

This application claims the priority benefit of China application serial no. 201610908808.9, filed on Oct. 19, 2016. The entirety of the above-mentioned patent application is hereby incorporated by reference herein and made a part of this specification.

### FIELD OF THE INVENTION

The invention relates to a sensor array, a manufacturing method thereof, and a sensing method; more particularly, the invention relates to a sensor array manufactured through applying a non-contact printing method, a manufacturing method thereof, and a sensing method.

### DESCRIPTION OF RELATED ART

Gas sensors can be applied to a variety of fields and contribute to maintenance of industrial security, detection of environmental pollution, or early diagnosis of diseases. According to the related art, in order to identify and detect various kinds of gases, a gas chromatograph (GC) and a mass spectrometry (MS) are often required to sense the gases. The GC and the MS are costly, lack portability, and must be operated by professionals; hence, the sensing process is cost- and time-consuming.

Besides, one single sensor does not have gas selectivity. To achieve gas selectivity, a gas separation system (e.g., a microchannel) is required to be arranged at the head of the sensor according to the related art, so as to identify different types of gases. Such a bulky sensor is detrimental to the miniaturization of sensors.

### SUMMARY OF THE INVENTION

The invention provides a sensor array, a manufacturing method thereof, and a sensing method, so as to provide a compact sensor array that contributes to the trend of miniaturization. Besides, the compact sensor array can be configured to directly sense a small amount of test samples of various kinds.

The invention further provides a sensor array, a manufacturing method thereof, and a sensing method, so as to integrate various kinds of sensing units for sensing a small amount of test samples of various kinds, and the methods provided herein are compatible to the conventional semiconductor manufacturing process.

The invention further provides a sensor array, a manufacturing method thereof, and a sensing method characterized by the increased selectivity of sensing materials and the miniaturized sensor array, so as to be used or applied in more and more fields.

In an embodiment of the invention, a sensor array includes a circuit board, a plurality of first sensing units, and at least one second sensing unit. The circuit board has an upper surface and a lower surface that are opposite to each other. The first sensing units are located on the upper surface of the circuit board. The first sensing units include a plurality of first electrodes and a plurality of sensing material layers. The sensing material layers are respectively located on surfaces of the first electrodes, and the sensing material layers are manufactured through applying a non-contact printing method. The second sensing unit is located on the upper surface of the circuit board. The second sensing unit includes a second electrode separated from the first electrodes. The sensing material layers respectively cover the surfaces of the first electrodes, and the second electrode is exposed to an atmospheric environment.

In an embodiment of the invention, a manufacturing method of a sensor array includes following steps. A circuit board is provided. The circuit board has an upper surface and a lower surface that are opposite to each other. A plurality of first electrodes and at least one second electrode are formed on the upper surface of the circuit board, wherein the first electrodes are separated from the at least one second electrode. A plurality of sensing material layers are respectively formed on surfaces of the first electrodes through applying a non-contact printing method but not formed on a surface of the at least one second electrode.

In an embodiment of the invention, a sensing method includes following steps. Mixed gases are sensed by the aforesaid sensor array. The sensing material layers in the sensor array are reacted with a plurality of gases of the mixed gases to generate a plurality of response signals. Parameter data are received from a reaction database, and concentrations of the gases are measured according to the parameter data and the response signals.

In view of the foregoing, the sensor array having a plurality of first sensing units is able to sense a small amount of test samples of various kinds because different sensing material layers in the sensor array can be reacted with different test samples. In addition, the at least one second sensing unit is sensing material-free and thus can be configured to sense the temperature in the atmospheric environment. That is, errors resulting from variations in the environmental temperature can be eliminated through temperature compensation, such that the accuracy of the resultant measured data is improved. In another aspect, the non-contact printing method can be applied to form a plurality of sensing material layers on the back surface of the circuit board and thus can have more selectivity of the sensing materials in comparison with the conventional semiconductor manufacturing process. The non-contact printing method can be integrated with the semiconductor manufacturing process, so as to increase the production speed. Moreover, the non-contact printing method can be applied to form the compact sensor array that contributes to the trend of miniaturization. In an embodiment of the invention, effects of gas selectivity can be accomplished without arranging any additional gas separation system. Different from the related art, an embodiment of the invention provides the miniaturized sensor array that can be used or applied in more and more fields and can comply with the requirements for commercialization of products.

To make the above features and advantages of the invention more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a top view illustrating a sensor array according to a first embodiment of the invention.

FIG. 2 is a schematic cross-sectional view illustrating a sensor array according to a second embodiment of the invention.

FIG. 3 is a schematic cross-sectional view illustrating a sensor array according to a third embodiment of the invention.

FIG. 4 is a schematic cross-sectional view illustrating a sensor array according to a fourth embodiment of the invention.

FIG. 5 shows the relationship between gas responses and gas concentrations according to the example 1.

FIG. 6 shows the relationship between gas responses and gas concentrations according to the example 2.

FIG. 7 shows the relationship between gas responses and gas concentrations according to the example 3.

### DESCRIPTION OF EMBODIMENTS

The invention described in several embodiments is elaborated with reference to the accompanying drawings. Note that the invention may be embodied in various ways and should not be limited to the embodiments provided herein. The thicknesses of layers and regions provided in the drawings are enlarged for illustrative purposes. The same or similar reference numbers represent the same or similar components and thus will not be described in each and every paragraphs below.

Although the sensor arrays 100, 200, 300, and 400 provided herein are exemplarily configured to sense gases, this should not be construed as a limitation to the invention. In other embodiments, the sensor arrays 100, 200, 300, and 400 can also be configured to sense light, humidity, temperature, or other environmental factors. Alternatively, the sensor arrays 100, 200, 300, and 400 can be configured to simultaneously sense gases, light, humidity, temperature, and other environmental factors.

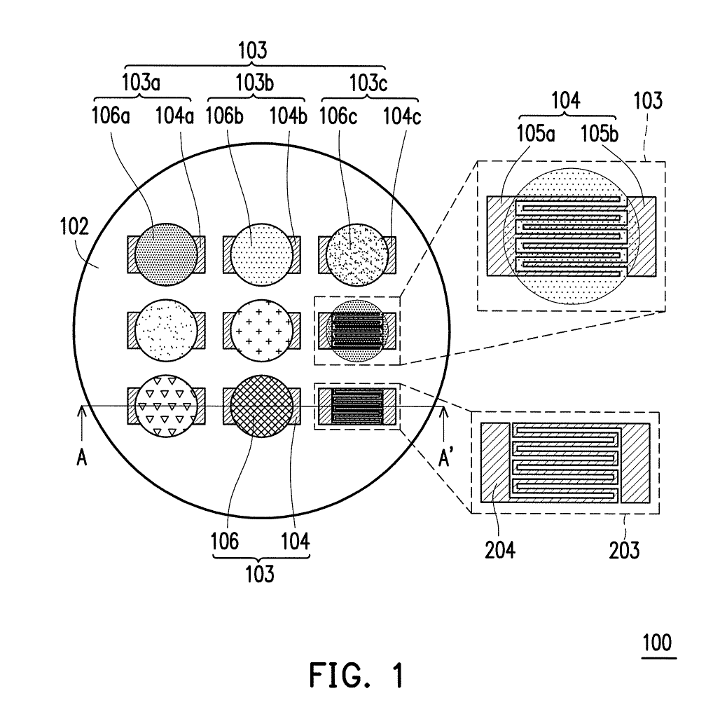

With reference to the top view of FIG. 1, the sensor array 100 provided in the first embodiment of the invention includes a circuit board 102, a plurality of first sensing units 103, and at least one second sensing unit 203. The circuit board 102 has a plurality of circuit layers and a plurality of dielectric layers (not shown) that are stacked to each other. In an embodiment of the invention, the circuit board 102 is a flexible circuit board, a rigid circuit board, or a rigid-flex circuit board, for example. The flexible circuit board may be equipped with a flexible dielectric layer and may include polyimide (PI), polyethylene terephthalate (PET), polythylene naphthalate (PEN), and so forth. The flexible circuit board is characterized by flexibility; namely, a surface of the flexible circuit board 102 may be a non-planar surface. The rigid circuit board may be equipped with a rigid dielectric layer and may include prepreg.

The first sensing units 103 are located on the circuit board 102. In an embodiment, the first sensing units 103 may be arranged in an array, for example. The first sensing units 103 are separated from each other and are not in contact with each other, so as to sense various types of test samples. In other words, the more the number of the first sensing units 130, the more the types of the test samples that can be sensed. According to an embodiment of the invention, the number of the first sensing units 103 may be greater than or equal to the types of the test samples. Besides, the sensing units in FIG. 1 are arranged in a 3×3 array (i.e., 8 first sensing units 103 and 1 second sensing unit 203). In other embodiments, the number of the first sensing units 103 may be determined according to actual needs.

Specifically, each of the first sensing units 103 includes a first electrode 104 and a sensing material layer 106. The first electrode 104 is located on the circuit board 102. In detail, each of the first electrodes 104 includes two sub-electrodes 105a and 105b. As shown in the enlarged view on the upper-right corner of FIG. 1, both the sub-electrodes 105a and 105b are interdigitated electrodes that are separated from each other and are not in contact with each other. The shape of the sub-electrodes 105a and 105b is not limited in the invention; as long as the sub-electrodes 105a and 105b are spaced apart from each other by a predetermined distance, are separated from each other, and are not in contact with each other, the sub-electrodes 105a and 105b fall within the scope of protection provided herein. In an alternative embodiment, the first electrodes 104 may also be stacked electrodes, for instance. The arrangement of the three-dimensional stacked electrodes effectively increases the intensity of the sensor array and reduce the overall volume of the device. Specifically, in the stacked electrodes, a plurality of electrode layers and a plurality of dielectric layers (not shown) may be vertically and alternately stacked onto the circuit board 102. Namely, at least one of the dielectric layers is arranged between two adjacent electrode layers, so as to electrically isolate the two adjacent electrode layers. In an embodiment of the invention, the electrode layers include a conductive material. The conductive material may be a doped or non-doped polysilicon material, a metallic material, or a combination thereof. The dielectric layers may be silicon oxide, silicon nitride, or a combination thereof.

In an embodiment, the sensing material layers 106 are respectively located on the first electrodes 104. Particularly, the sensing material layers 106 cover surfaces of the sub-electrodes 105a and 105b and fill the space between the sub-electrodes 105a and 105b. Although the sensing material layers 106 shown in FIG. 1 do not cover all surfaces of the sub-electrodes 105a and 105b (or the first electrodes 104). In other embodiments of the invention, the sensing material layers 106 may cover all surfaces (including the top and side surfaces) of the sub-electrodes 105a and 105b (or the first electrodes 104). If the test samples are attached to or in contact with the surfaces of the sensing material layers 106, the test samples can be reacted with the sensing material layers 106, such that capacitances, resistances, or other electrical properties of the sensing material layers 106 between the sub-electrodes 105a and 105b are changed.

For instance, the sensor array 100 is a gas sensor array. As shown in FIG. 1, the sensor array 100 at least includes three first sensing units 103a, 103b, and 103c. If the test samples are mixed gases having three types of gases, the three types of gases in the mixed gases are reacted with the sensing material layers 106a, 106b, and 106c, such that the capacitances, the resistances, or other electrical properties of the sensing material layers 106a, 106b, and 106c are changed. The data of the changed capacitances, the resistances, or other electrical properties can then be transmitted to the circuit board 102 through the first electrodes 104a, 104b, and 104c below the sensing material layers 106a, 106b, and 106c for subsequent data processing. Thereby, the sensor array 100 provided in the present embodiment is able to simultaneously sense three different kinds of gases in no need of being equipped with any additional gas separation system. Besides, if the sensor array 100 is an ultraviolet light sensor array, the ultraviolet light can be reacted with the sensing material layers 106, so as to change the resistances of the sensing material layers 106. As such, the sensor array 100 is able to detect whether the strength of the ultraviolet light in the environment is excessive and thereby remind users of blocking sunlight or wearing sunscreens.

Note that the sensing material layers 106 may be formed by applying a non-contact printing method, for instance. According to an embodiment of the invention, the non-contact printing method includes an ink jet printing method or an aerosol jet printing method. In the aerosol jet printing method, for instance, an aerosol jet deposition head is applied to form an annularly propagating jet constituted by an outer sheath flow and an inner aerosol-laden carrier flow. During an annular aerosol jet process, an aerosol stream having the sensing materials is focused and deposited onto the planar or non-planar circuit board 102. The sensing material layers 106 are then formed on the first electrodes 104 after thermal treatment or photochemical treatment. Said steps may be referred to as maskless mesoscale material deposition (M3D); that is, the deposition process can be performed without using any mask, and the deposited material layers can have the linewidth within the range from 1 μm to 10 μm.

According to an embodiment of the invention, the size of the resultant sensing material layers 106 or an area occupied by the sensing material layers 106 is within a range from 1 μm² to 10⁶ μm², e.g., 10 μm². In response to the decrease in the size of the sensing material layers 106, the size of the sensor array 100 having the sensing material layers 106 can be reduced to 1 μm² to 10⁶ μm². Compared to the conventional sensor array (whose size is about 10⁸ μm²), the sensor array 100 provided herein is compact and can be applied to portable electronic apparatuses with the reduced size, such as mobile phones, tablet PCs, music players, a combination thereof, or the like.

In addition, through applying the non-contact printing method provided in the present embodiment, materials (e.g., gold nanoclusters, magnetic materials, or biomimetic organic material) that are incompatible with the semiconductor manufacturing process can be formed on the circuit board 102. Hence, the selectivity of the sensing materials is greater in the process provided in the present embodiment than in the conventional semiconductor manufacturing process. Specifically, except for metal and metal oxide, most sensing materials cannot be formed on the sensor array through applying the conventional semiconductor manufacturing process. The non-contact printing method provided in the present embodiment not only can be applied to form various kinds of sensing materials (that are compatible or incompatible with the semiconductor manufacturing process) in the sensor array but also can be integrated with the semiconductor manufacturing process; thereby, the production can be accelerated, and the requirement for commercialization of products can be satisfied. Besides, the conventional manufacturing method can merely be applied to form the sensing materials on planes; by contrast, the non-contact printing method provided herein allows the sensing material layers 106 to be formed on curved surfaces, concave surfaces, inclined surfaces, surfaces having a combination of said surfaces, or similar surfaces, which cannot be done by performing the conventional manufacturing method.

In an embodiment of the invention, the sensing material layers 106 include metal, metal oxide, graphene, graphene oxide, carbon nanotubes, fullerene, gold clusters, polymers, metal sulfides, quantum dots, calcium titanium ore, or a combination thereof. Metal may be nickel, copper, or any other appropriate metallic material, for instance. Metal oxide may be zinc oxide, tin oxide, tungsten oxide, magnesium oxide, titanium oxide, iron oxide, zirconium oxide, or any other appropriate material, for instance. Polymers may be poly-3, 4-ethylenedioxythiophene (PEDOT) or any other appropriate material, for instance.

Please refer to FIG. 1. The sensor array 100 provided in the first embodiment of the invention includes the second sensing unit 203 located on the circuit board 102. The second sensing unit 203 includes a second electrode 204. As shown in the enlarged view on the lower-right corner in FIG. 1, the second electrode may be a serpentine electrode, for instance. The so-called serpentine electrode means that the electrode is arranged spirally between two points, so as to reduce the occupied area and increase the effective surface area. In other embodiments, the second electrode 204 may also be of other shapes.

It should be mentioned that data should be adjusted according to the humidity and the temperature in the environment during the aforesaid gas sensing process. The humidity sensing process can be performed by forming a moisture sensing material on one of the first electrodes 104. The temperature sensing process can be performed by exposing the second electrode 204 to the atmospheric environment, so as to sense the temperature in the atmospheric environment. Namely, the second sensing unit 203 is sensing material-free. In particular, the second sensing unit 203 senses the temperature in the atmospheric environment on the premise that the resistance of the second electrode 204 (which is not covered by any sensing material) is changed in response to changes to the temperature in the environment. Compared to the temperature sensors on the market, the second electrode 204 (or the second sensing unit 203) provided in the present embodiment is more sensitive, occupies a smaller area, and requires a lower manufacturing cost; additionally, the second electrode 204 (or the second sensing unit 203) can be formed on various types of base materials through applying the printing method. Accordingly, the second electrode 204 (or the second sensing unit 203) provided in the present embodiment can be extensively applied to various electronic devices.

For instance, if the sensing material is metal oxide, for instance, the moisture in the environment is attached to the surface of the metal oxide, and thus an additional conductive channel is formed. Thereby, the resistance is reduced, and the equivalent capacitance is increased. That is, the greater the humidity is, the more the resistance is reduced, and the more the capacitance is increased. If the temperature in the environment is changed, the increase in the temperature leads to the reduction of the resistance of the metal oxide, and the decrease in the temperature leads to the increase in the resistance of the metal oxide. Hence, the temperature and the humidity can be considered as the base electrical level of the sensor array. In other words, the sensor array 100 provided herein can be applied to additionally sense the humidity and the temperature in the environment, so as to ensure the accuracy of the gas sensing data.

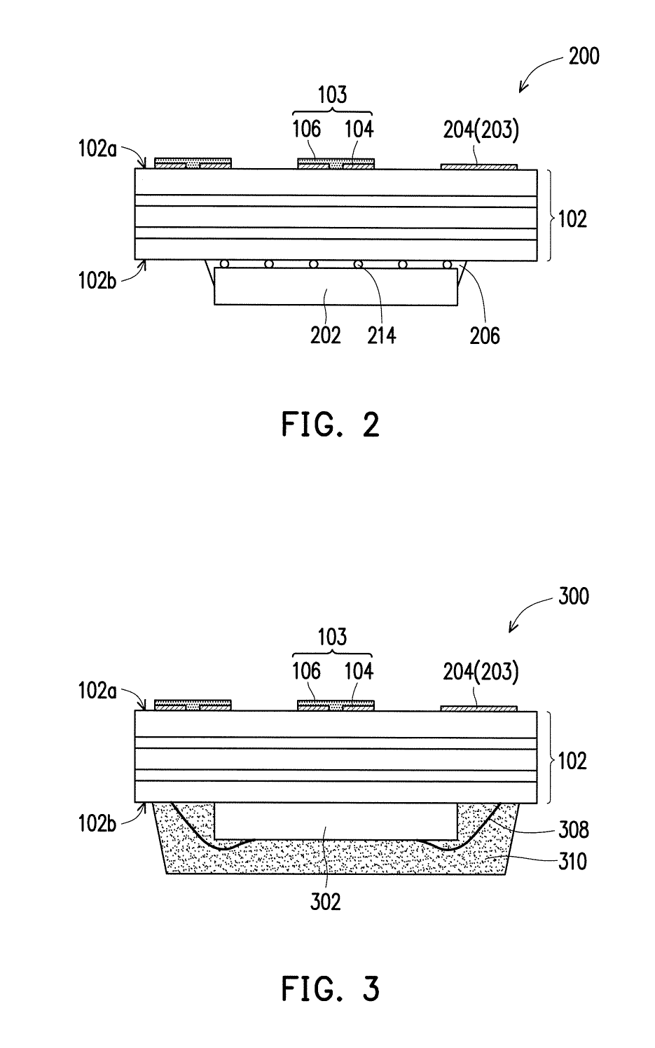

Please refer to FIG. 2. FIG. 2 is, for instance, a schematic cross-sectional view taken along a sectional line A-A′ depicted in FIG. 1. The sensor array 200 provided in the second embodiment of the invention includes a circuit board 102, a plurality of first sensing units 103, and at least one second sensing unit 203. The circuit board 102 has an upper surface 102a and a lower surface 102b that are opposite to each other. In an embodiment of the invention, the upper surface 102a of the circuit board 102 may be the back surface of the circuit board 102, and the lower surface 102b of the circuit board 102 may be the front surface of the circuit board 102. The first sensing units 103 and the second sensing unit 203 are located on the upper surface 102a of the circuit board 102. The first sensing units 103 include a plurality of first electrodes 104 and a plurality of sensing material layers 106. The sensing material layers 106 cover surfaces of the first electrodes 104 and fill the space between the first electrodes 104. The sensing material layers 106 shown in FIG. 2 do not cover the side surfaces of the first electrodes 104. In other embodiments of the invention, the sensing material layers 106 may completely cover the top and side surfaces of the first electrodes 104. The second sensing unit 203 includes a second electrode 204. The second sensing unit 203 does not have any sensing material that covers the surface of the second electrode 204. Since the materials of the circuit board 102, the first sensing units 103, and the second sensing units 203 depicted in FIG. 2 are similar to those of the circuit board 102, the first sensing units 103, and the second sensing units 203 depicted in FIG. 1 and have been described in the previous paragraphs, no further explanation is provided below.

In the second embodiment, the sensor array 200 further includes a chip 202 that is located on the lower surface 102b of the circuit board 102. The chip 202 can be electrically connected with the circuit board 102 through flip-chip bonding. Through the so-called flip-chip bonding, the chip 202 is electrically connected to the circuit board 102 via a plurality of bumps 214 between the circuit board 102 and the chip 202. Besides, an underfill 206 fills the space between the circuit board 102 and the chip 202, so as to encapsulate the bumps 214.

According to an embodiment of the invention, the chip 202 may be a micro control unit (MCU), a Bluetooth chip, or any other appropriate chip, for instance. The chip 202 can receive the data (i.e., the data including the changes to the capacitances, the resistances, or other electrical properties of the sensing material layers 106 and the changes to the resistance of the second electrode 204) measured or sensed by the first sensing units 103 and the second sensing unit 203 and process or transmit the received data. Although FIG. 2 illustrates only one chip 202, the invention should not be not limited thereto. In other embodiments, the number and the type of the chip 202 may be adjusted according to actual needs.

Please refer to FIG. 3, which is a schematic cross-sectional view taken along the sectional line A-A′ depicted in FIG. 1, for instance. The sensor array 300 provided in the third embodiment is similar to the sensor array 200 provided in the second embodiment, while the difference between the sensor arrays 300 and 200 lies in that the sensor array 300 is electrically connected to the circuit board 102 through wire bonding. Through the so-called wire bonding, the chip 302 is electrically connected to the circuit board 102 via a plurality of conductive wires 308. An encapsulant 310 is then applied to cover the chip 302 and a portion of the lower surface 102b of the circuit board 102 and encapsulate the conductive wires 308.

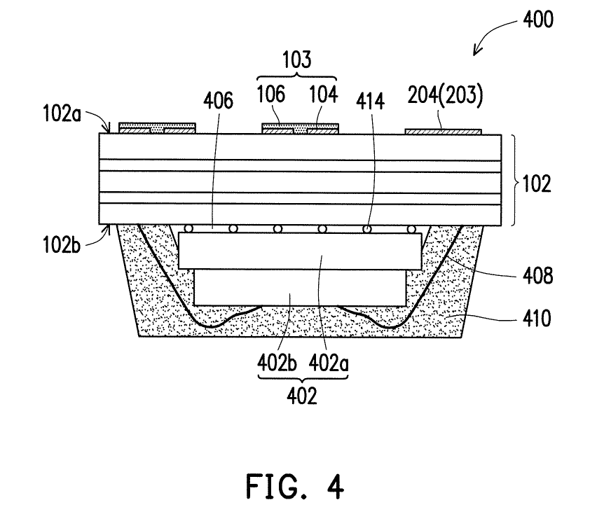

Please refer to FIG. 4, which is a schematic cross-sectional view taken along the sectional line A-A′ depicted in FIG. 1, for instance. The sensor array 400 provided in the fourth embodiment is similar to the sensor array 200 provided in the second embodiment, while the difference between the sensor arrays 400 and 200 lies in that the sensor array 400 has the stacked chip structure 402 that includes chips 402a and 402b that are stacked. The chip 402a is located between the circuit board 102 and the chip 402b. The chip 402a is electrically connected to the circuit board 102 through flip-chip bonding. That is, the chip 402a is electrically connected to the circuit board 102 via the bumps 414. An underfill 406 then fills the space between the circuit board 102 and the chip 402a, so as to encapsulate the bumps 414. The chip 402b is electrically connected to the circuit board 102 through wire bonding. Through the so-called wire bonding, the sensor array 400 is electrically connected to the circuit board 102 and the chip 402b via the conductive wires 408. An encapsulant 410 is then applied to cover the chips 402a and 402b, the underfill 406, and a portion of the lower surface 102b of the circuit board 102 and encapsulate the conductive wires 408. Although FIG. 4 illustrates only two chips 402a and 402b in the stacked chip structure 402, the invention should not be not limited thereto. In other embodiments, the number and the type of the chips may be adjusted according to actual needs.

In an embodiment of the invention, a sensing method is also provided and includes following steps. Mixed gases are sensed by any of the sensor arrays 100, 200, 300, and 400 (referred to as the sensor arrays 100-400 hereinafter). The sensing material layers 106 in the sensor arrays 100-400 are reacted with a plurality of gases of the mixed gases to generate a plurality of response signals. Parameter data are received from a reaction database, and concentrations of the gases are measured according to the parameter data and the response signals. In particular, a method of measuring the concentrations of the gases according to the parameter data and the response signals is described below. The parameter data and the response signals are substituted into a formula 1 to obtain the concentrations of the gases. The formula 1 is

$$[ \begin{matrix}{R}_{w} \\ {R}_{t} \\ {R}_{z}\end{matrix} ] = [ \begin{matrix}{S}_{wm} & {S}_{we} & {S}_{wt} \\ {S}_{tm} & {S}_{te} & {S}_{tt} \\ {S}_{zm} & {S}_{ze} & {S}_{zt}\end{matrix} ] [ \begin{matrix}{C}_{m} \\ {C}_{e} \\ {C}_{t}\end{matrix} ]$$

Here, R_{w}, Rₜ, and R_{z} are the response signals, S_{wm}, Sₜₘ, S_{zm}, S_{we}, Sₜₑ, S_{ze}, S_{wt}, Sₜₜ, and S_{zt} are the parameter data, and Cₘ, Cₑ, and C₁ are the concentrations of the gases.

In the present embodiment, the second electrode 204 (or the second sensing unit 203) in each of the sensor arrays 100-400 can be applied to sense the temperature in the atmospheric environment, so as to adjust the response signals and ensure the accuracy of the obtained concentration of the gases. In other words, the sensor arrays 100-400 provided herein can be configured to simultaneously sense gases as well as other environmental factors including humidity and temperature, so as to eliminate the impact of the environmental factors including humidity and temperature and ensure the accuracy of the measured data.

According to an embodiment of the invention, the gases include volatile organic compounds or inorganic gases. The volatile organic compounds may be alkane, aromatic hydrocarbons, alkene, halohydrocarbon, esters, aldehydes, ketones, or a combination thereof, for instance. The inorganic gases may be carbon monoxide, carbon dioxide, ammonia, nitric monoxide, nitric dioxide, hydrogen sulfide, or a combination thereof.

To prove the feasibility of the invention, several examples are provided hereinafter to further elaborate the sensor arrays.

### Examples 1-3

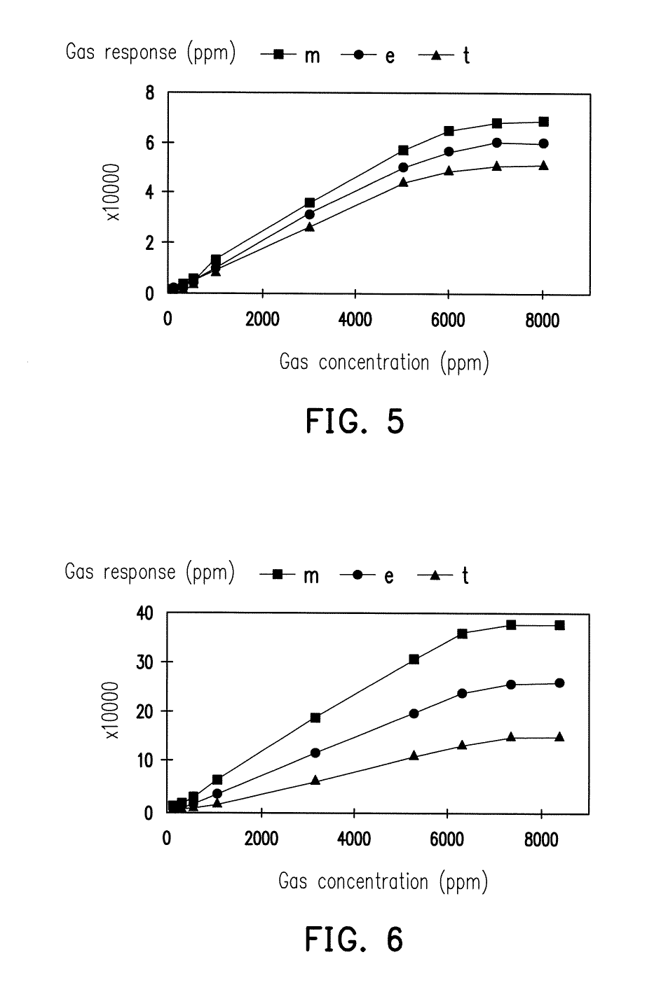

The sensor array depicted in FIG. 2 is taken for example, and tungsten oxide (example 1), titanium oxide (example 2), and zinc oxide (example 3) are considered as the sensing material layers to form three sensing units. Mixed gases containing methanol m, ethanol e, and toluene t are reacted with the sensing material layers in the examples 1-3, so as to generate the gas response signals R_{w}, Rₜ, and R_{z}. The results are shown in FIG. 5-FIG. 7.

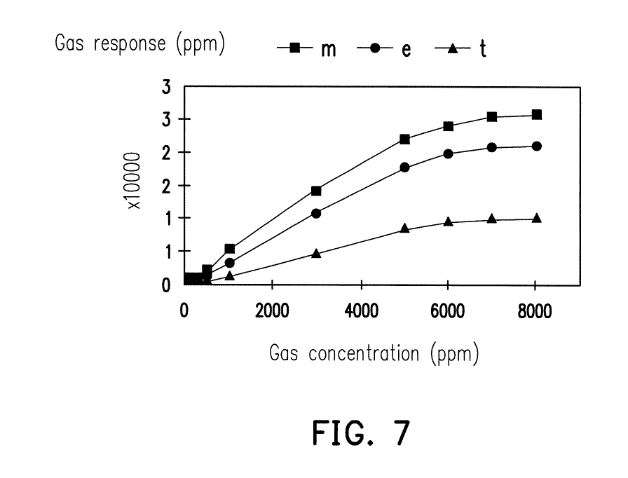

FIG. 5 shows the relationship between gas responses and gas concentrations according to the example 1. FIG. 6 shows the relationship between gas responses and gas concentrations according to the example 2. FIG. 7 shows the relationship between gas responses and gas concentrations according to the example 3.

As shown in FIG. 5-FIG. 7, the gas concentrations are 0-6000 ppm, and there is a linear relationship between the gas responses and the gas concentrations. That is, the reactions of the mixed gases and the sensing material layers in the examples 1-3 lead to the gas response signals R_{w}, Rₜ, and R_{z}, and the parameter data of the mixed gases (i.e., methanol m, ethanol e, and toluene t) are obtained from the reaction database, so as to list three simultaneous equations. Said three simultaneous equations can be applied to obtain three unknown numbers, so as to obtain the concentrations of the methanol m, the ethanol e, and the toluene t in the mixed gases.

Said three simultaneous equations can be represented by formula 1. The formula 1 is

$$[ \begin{matrix}{R}_{w} \\ {R}_{t} \\ {R}_{z}\end{matrix} ] = [ \begin{matrix}{S}_{wm} & {S}_{we} & {S}_{wt} \\ {S}_{tm} & {S}_{te} & {S}_{tt} \\ {S}_{zm} & {S}_{ze} & {S}_{zt}\end{matrix} ] [ \begin{matrix}{C}_{m} \\ {C}_{e} \\ {C}_{t}\end{matrix} ]$$

Here, R_{w}, Rₜ, and R_{z} are the response signals generated by the reactions of the mixed gases and the sensing material layers in the examples 1-3, S_{wm}, Sₜₘ, S_{zm}, S_{we}, Sₜₑ, S_{ze}, S_{wt}, Sₜₜ, and S_{zt} are the parameter data, and Cₘ, Cₑ, and Cₜ are the concentrations of the gases of the methanol m, the ethanol e, and the toluene t.

To sum up, the sensor array having a plurality of first sensing units is able to sense a small amount of test samples of various kinds because different sensing material layers in the sensor array can be reacted with different test samples. In addition, the at least one second sensing unit is sensing material-free and thus can be configured to sense the temperature in the atmospheric environment. That is, errors resulting from variations in the environmental temperature can be eliminated through temperature compensation, such that the accuracy of the resultant measured data is improved. In another aspect, the non-contact printing method can be applied to form a plurality of sensing material layers on the back surface of the circuit board. Hence, the non-contact printing method provided herein can have more selectivity of the sensing materials in comparison with the conventional semiconductor manufacturing process and can be integrated with the semiconductor manufacturing process to expedite the production. Moreover, effects of gas selectivity can be accomplished without arranging any additional gas separation system. Different from the related art, an embodiment of the invention provides the miniaturized sensor array that can be used or applied in more and more fields and can comply with the requirements for commercialization of products.

What is claimed is:

1. A sensor array comprising:
a circuit board having an upper surface and a lower surface opposite to each other;
a plurality of first sensing units located on the upper surface of the circuit board and comprising:
a plurality of first electrodes; and
a plurality of sensing material layers respectively located on surfaces of the first electrodes, wherein the sensing material layers are manufactured through applying a non-contact printing method; and
at least one second sensing unit located on the upper surface of the circuit board and comprising a second electrode separated from the first electrodes, wherein the sensing material layers respectively cover the surfaces of the first electrodes, and the at least one second sensing unit is exposed to an atmospheric environment.

2. The sensor array according to claim 1, wherein the sensing material layers comprise metal, metal oxide, graphene, graphene oxide, carbon nanotubes, fullerene, gold clusters, polymers, metal sulfides, quantum dots, calcium titanium ore, or a combination thereof.

3. The sensor array according to claim 1, further comprising a chip located on the lower surface of the circuit board, the chip being electrically connected to the circuit board through wire bonding or flip-chip bonding.

4. The sensor array according to claim 1, further comprising a plurality of chips located on the lower surface of the circuit board, the chips being stacked together to constitute a stacked chip structure.

5. The sensor array according to claim 1, wherein the first electrodes comprise interdigitated electrodes, stacked electrodes, or a combination thereof, and the first sensing units are configured to sense gas, light, humidity, or a combination thereof.

6. The sensor array according to claim 1, wherein the second electrode is a serpentine electrode, and the at least one second sensing unit is configured to sense temperature.

7. The sensor array according to claim 6, wherein the at least one second sensing unit is sensing material-free.

8. The sensor array according to claim 1, wherein the upper surface or the lower surface of the circuit board is a curved surface, a concave surface, an inclined surface, or a surface having a combination of said surfaces.

9. The sensor array according to claim 1, wherein an area occupied by the sensing material layers is within a range from 1 μm² to 10⁶ μm², and an area occupied by the sensor array is within a range from 1 μm² to 10⁶ μm².

10. A manufacturing method of a sensor array, comprising:
providing a circuit board having an upper surface and a lower surface opposite to each other;
forming a plurality of first electrodes and at least one second electrode on the upper surface of the circuit board, wherein the first electrodes are separated from the at least one second electrode; and
respectively forming a plurality of sensing material layers on surfaces of the first electrodes through applying a non-contact printing method but not on a surface of the at least one second electrode.

11. The manufacturing method according to claim 10, wherein the non-contact printing method comprises an ink jet printing method or an aerosol jet printing method.

12. A sensing method comprising:
sensing mixed gases by the sensor array according to claim 1, wherein the sensing material layers in the sensor array are reacted with a plurality of gases of the mixed gases to generate a plurality of response signals; and
receiving parameter data from a reaction database and measuring concentrations of the gases according to the parameter data and the response signals.

13. The sensing method according to claim 12, wherein the step of measuring the concentrations of the gases according to the parameter data and the response signals comprises:
substituting the parameter data and the response signals into a formula 1 to obtain the concentrations of the gases, wherein the formula 1 is
$$[ \begin{matrix}{R}_{w} \\ {R}_{t} \\ {R}_{z}\end{matrix} ] = [ \begin{matrix}{S}_{wm} & {S}_{we} & {S}_{wt} \\ {S}_{tm} & {S}_{te} & {S}_{tt} \\ {S}_{zm} & {S}_{ze} & {S}_{zt}\end{matrix} ] [ \begin{matrix}{C}_{m} \\ {C}_{e} \\ {C}_{t}\end{matrix} ] ,$$
R_{w}, Rₜ, and R_{z}, are the response signals, S_{wm}, Sₜₘ, S_{zm}, S_{we}, Sₜₑ, S_{ze}, S_{wt}, Sₜₜ, and S_{zt} are the parameter data, and Cₘ, Cₑ, and Cₜ are the concentrations of the gases.
